SEQUENCE LISTING (3 sequences)

SEQ ID NO: 1 - protein, 330 aa, unidentified
  source 1..330: note = Bacteria
MPLKNRLLARLSCVAAVVAATAAVAPLTLVSTAHAAAPQVRTSAPGYYRMLLGDFEITALSDGTVALPVDKRLNQPAPKTQSALAKYFQKAPLETSVTGYLVNTGSKLVLVDTGAAGLFGPTLGRLAANLKAAGYQPEQVDEIYITHMHPDHVGGLMVGEQLAFPNAVVRADQKEADFWLSQTNLDKAPDDSKGFFKGAMASLNPYVKAGKFKPFSGNTDLVPGIKALASHGHTAGHTTYVVESQGQKLALLGDLILVAAVQFDDPSVTNQLDSDSKSAAVERKKAFADAAKGGYLIAAAHLSFPGIGHIRAEGKGYRFVPVNYSVVNPK

SEQ ID NO: 2 - protein, 491 aa, unidentified
  source 1..491: note = Bacteria
MERSDLDYASATEIARLVRTRQISAADVTEHAISRIEARNGSLNAFVYTDFEQARSRAKDLDTRISAGEDVGPLAGVPTAIKDLFNFYPGWPSTLGGIRCLRDFKLDVKSRYATKMEEAGAVVLGITNSPVLGFRGTTDNDLYGPTRNPFDLSRNSGGSSGGTSAAVADGLLPIGDGTDGGGSIRIPAAWCHVFGFQASPGRIPLAIRPNAFGAAAPFIYEGPITRTVEDAALAMSVLAGSDPADPFSLNDRLDWLGAVDQPITSLRIGFTPDFGGFPVEPAVAATIAHAVRAFEQAGAKIVPLKLDFGYTHDELSQLWCRMISQGTIAVVDSFAENGLHLEPDFPAPVMEWAQKAKNATPLDLHRDQVMRTKVYDVLNAAFSQVDLIAGPTTTCLPTPNGERGMTVGPSEIAGTPINRLIGFCPTFLTNFTGNPAASLPAGLADGLPVGLMLIGPRRDDLTVLSASAAFERVQPWADSYRIPAARPLGSQ

SEQ ID NO: 3 - protein, 247 aa, unidentified
  source 1..247: note = Bacteria
MMKLASPKPFTFEGGDRAVLLLHGFTGNSADVRMLGRFLEKKGYTCHAPIYKGHGVPPEELVHTGPEDWWQDVTEAYQLLKDKGFEKIAVVGLSLGGVFSLKLGYTVPVLGVVPMCAPMYIKSEETMYQGILAYAREYKKREQKSPEQIEQEMLEFQKTPMNTLKALQQLIADVRNNVDMIYAPTFVVQARHDEMINTDSANIIYNGVESTLKDIKWYEDSTHVITLDKQRDELHEDVYNFLEQLDW

(19) United States
(12) Patent Application Publication
(10) Pub. No.: US 2025/0361495 A1
(43) Pub. Date: Nov. 27, 2025

(54) RECOMBINANT PLASMID VECTORS, METHOD FOR PRODUCING ENZYMES THAT HYDROLYZE ORGANOPHOSPHATE, CARBAMATE, AND PYRETHROID INSECTICIDES, AND THE FORMULATION OF SUCH ENZYMES AS FUNCTIONAL COMPONENTS

(71) Applicant: BIOM CO., LTD., Nonthaburi (TH)

(72) Inventor: Alisa VANGNAI, Bangkok (TH)

(21) Appl. No.: 18/859,512

(22) PCT Filed: Apr. 24, 2023
(86) PCT No.: PCT/IB2023/054170

§ 371 Date: Oct. 23, 2024

(30) Foreign Application Priority Data
Apr. 25, 2022 (TH) 2201002469

(51) Int. Cl. C12N 9/18; C12N 9/48; C12N 15/52; C12N 15/70

(52) U.S. Cl. CPC C12N 9/18; C12N 9/485; C12N 15/52; C12N 15/70; C12N 2800/40; C12Y 301/01088; C12Y 301/08001; C12Y 304/11019

(57) ABSTRACT

A process for producing three recombinant enzymes capable of hydrolyzing each class of organophosphate, carbamate, and pyrethroid insecticides, as well as a formulation containing these enzymes provides the recombinant plasmid DNA vector, in which the protein secretory system has been modified to enhance the secretion of recombinant protein into the periplasmic space or culture medium. A nucleotide sequence encoding PelB (pectate lyase B) signal sequence for periplasmic localization is deleted and replaced with a nucleotide sequence encoding LamB (maltoporin or phage lambda receptor) signal sequence to enable a more efficient secretory recombinant protein. Three recombinant plasmid DNA vectors for the expression of recombinant proteins that are constructed using the modified plasmid DNA vector described above. The recombinant proteins include a recombinant organophosphate-hydrolyzing enzyme; a recombinant carbamate-hydrolyzing enzyme; and a recombinant pyrethroid-hydrolyzing enzyme.

### CROSS REFERENCE TO RELATED APPLICATIONS

This application is a National Stage Application of International Application PCT/IB2023/054170, filed on Apr. 24, 2023 and claims priority of application No. 2201002469 filed in Thailand on Apr. 25, 2022 under 35 U.S.C. § 119; the entire contents of all of which are hereby incorporated by reference.

### REFERENCE TO AN ELECTRONIC SEQUENCE LISTING

The content of the electronic sequence listing (Substitute Sequence Listing.mxl; Size: 5,360 bytes; and Date of Creation: Aug. 7, 2025) is herein incorporated by reference in its entirety.

### TECHNICAL FIELD

The present invention relates to the field of biotechnology, particularly enzyme biotechnology.

### BACKGROUND

Pesticides, including insecticides, are synthetic chemicals extensively used in modern agriculture to eliminate pests during crop production and post-harvest, in order to maintain crop yield and quality. In addition, they are used to prevent insect infestation in some types of dried foods, such as dried fish, beef jerky, etc. The intensive use of these hazardous chemicals in agriculture leads to their contamination and accumulation in agricultural soil and water, crop produce, and foods, thereby posing a threat to the health of farmers and consumers. Long-term insecticide exposure through consumption and accumulation is harmful to humans, even at low concentrations, because these insecticides can cause acute and/or chronic toxicity. Acute toxicity symptoms include irritation to the respiratory system, digestive system, nervous system, and muscles. Chronic toxicity symptoms comprise adverse effects on the liver, kidneys, immune system, and endocrine glands (hormone system); genetic alterations and mutations that may lead to cancer; teratogenic effects; and arrested brain development in infants and young children. Furthermore, the rejection of agricultural products with excessive insecticide residues by trading partner countries has negatively impacted the export businesses and economies of relevant exporting nations.

Several criteria are used to classify pesticides. Pesticides can be categorized into [1] 8 groups according to their form or compound state, e.g. Water Dispersible Powder (WDP), Emulsifiable Concentrate (EC), and Suspension Concentrate (SC) [Lin et al. (2023) Microorganisms, 11, 364]; [2] at least 29 groups according to their mechanism of action, e.g. acetylcholinesterase inhibitor, which affects the nervous system, causes sodium balance disturbance, and acts as a chloride channel blocker [Insecticide Resistance Action Committee, IRAC (2023): irac-online.org]; and [3] 4 groups according to their chemical type or chemical structure, namely (i) organophosphates, which are compounds containing a phosphorus moiety as a key substituent, (ii) carbamates that consist of compound derivatives of carbamic acid, (iii) pyrethroids, which are synthetic derivatives of naturally occurring pyrethrin, and (iv) organochlorines, constituting compounds that contain at least one carbon-chlorine bond [Zacharia, J. T. (2011) ISBN 978-953-51-5154-8]. Due to the high toxicity of organochlorines to humans and their high environmental persistence, they are banned in most countries.

Organophosphate, carbamate and pyrethroid insecticides are extensively used in both agriculture and residential settings. Reports have indicated the presence of predominantly organophosphate and carbamate residues, in quantities exceeding the legal limit, on random samples of vegetables and fruit from fresh markets and supermarkets, including kale, morning glory [Wanwimolruk et al. (2015) Environ Health Prev Med 20 (3), 204; Wanwimolruk et al. (2016) PeerJ 4, 2432], mangosteens [Phopin et al. (2017) J Sci Food Agric 97, 832], and mushrooms [Phutphat et al. (2016)]. Organophosphate and carbamate insecticides are potent inhibitors of acetylcholinesterase, an enzyme that hydrolyzes acetylcholine and plays an important role in the normal functioning of the central nervous system of humans and mammals. Exposure to these insecticides at low concentrations results in nausea, dizziness, vomiting, cramps, convulsions, respiratory failure, sympathetic overstimulation, and skeletal muscular paralysis, or unconsciousness. Death may occur within 24 hours, when the subject is exposed to organophosphates and carbamates at high concentrations and left untreated. Long-term exposure may cause brain cancer and leukemia.

The presence, persistence, and bioaccumulation of insecticide residues in the environment cause severe adverse impacts not only on humans and living organisms in the food chain, but also on the ecosystem. This results in the pollution of soil, water, and sediments. The soil pollution in agricultural areas brings about soil infertility and has detrimental effects on plant growth and development as well as yield. Moreover, the contamination of crop produce with these residues, especially in concentrations exceeding standard limits, often induces the rejection of such produce at border inspection posts and economic loss.

Current methods for cleaning fruits and vegetables show differences in pesticide elimination effectiveness. The Agricultural Pesticide Division at the Department of Agriculture in the Ministry of Agriculture and Cooperatives of Thailand reported that pesticide residues in fruits and vegetables decreased by 25-39 percent after a two-minute wash with running water, 48-50 percent with heat, and 22-36 percent when soaked in a chemical detergent for 10 minutes, followed by a wash under running water [https://shorturl.at/dtJ) V7]. However, these washing techniques do have some shortcomings. The washing of fruits and vegetables with running water must be performed for a long duration and requires a large quantity of water that cannot be reused. In contrast, the residues of chemicals used to wash fruits and vegetables may be harmful to consumers or negatively impact the quality of said agricultural products. For instance, exposure to high levels of sodium bicarbonate residues from chemical washing, particularly in factories, may cause severe headaches, diarrhea, nausea or vomiting, irritability, and restricted breathing. Potassium permanganate and chlorine vapors may even irritate the factory operator's nose, throat, and lungs upon inhalation, leading to coughing and/or shortness of breath, whereas exposure to the residues of such chemicals harms the digestive system and may cause blindness, if they come into contact with the eyes. Certain washing techniques also diminish crop produce quality. For example, washing fruits and vegetables with vinegar induces withering, while imbuing said produce with an undesirable flavor and pungent odor. In addition, heating, steaming, or boiling fresh fruits and vegetables in water would only relieve them of their nutritional value. Therefore, this method would not be appropriate for exporting fresh crop produce.

Besides physical and chemical treatments, pesticides are effectively degraded by enzyme-catalyzed biological reactions. Enzymes are natural biomolecules of protein that are harmless for both humans and the environment. Hence, insecticide-degrading enzymes can be used as functional components in fruit and vegetable washing agents to minimize insecticide residues on both pre-harvested and post-harvested fruits and vegetables. Furthermore, they can be applied as bioremediating agents to treat pesticide-contaminated groundwater and surface water, for the purpose of improving the quality of drinking water supplies.

According to scientific studies, various microbial enzymes are capable of catalyzing the hydrolysis of organophosphate, carbamate, and pyrethroid insecticides [Bhatt et al (2021) J Haz Mat 411 (5), 125026]. These enzyme catalytic activities occur naturally in microbial metabolism, such as that occurring in bacteria, in order to transform and detoxify pesticide molecules and metabolize them as carbon and energy sources. Nevertheless, these insecticide-degrading enzymes have diverse catalytic properties and efficiencies, and the majority of them are intracellular enzymes, which limits their application.

The heterologous gene expression in a bacterial host cell (e.g. Escherichia coli and Bacillus subtilis) and the methods of transformation have been disclosed in the prior art [Kataoka et al (2011) AMB Express 1 (1), 1; Kataoka et al (2013) J Biosci Bioeng 115 (5), 475; Mahipant et al. (2019) J Gen Appl Microbiol 65, 96]. In general, recombinant host cells must meet important criteria, such as producing the protein or enzyme of interest in high or acceptable yields, being capable of efficient secretion of the protein or enzyme, and being simple to manipulate and maintain.

This present embodiment relates to a process of producing recombinant enzymes, each of which is capable of hydrolyzing organophosphate, carbamate, and pyrethroid insecticides, and the formulation thereof. It further involves the construction of plasmid DNA vectors for extracellular expression of insecticide-hydrolyzing enzymes in a recombinant host cell that is Escherichia coli strain BL21 (DE3), in order to use these enzymatic cocktails as functional components, in required proportions, for the formulation of the final product.

### SUMMARY OF INVENTION

The present embodiment relates to a process for producing three recombinant enzymes capable of hydrolyzing each class of organophosphate, carbamate, and pyrethroid insecticides, as well as a formulation containing these enzymes.

The present embodiment provides the recombinant plasmid DNA vector, in which the protein secretory system has been modified to enhance the secretion of recombinant protein into the periplasmic space or culture medium. A nucleotide sequence encoding PelB (pectate lyase B) signal sequence for periplasmic localization was deleted and replaced with a nucleotide sequence encoding LamB (maltoporin or phage lambda receptor) signal sequence to enable a more efficient secretory recombinant protein.

The present embodiment discloses three recombinant plasmid DNA vectors for the expression of recombinant proteins that were constructed, using the modified plasmid DNA vector described above. The recombinant proteins include the following:
(a) A recombinant organophosphate-hydrolyzing enzyme having at least 80 percent sequence identity to the amino acid sequence of methyl parathion hydrolase (MPH) (Protein Database (PDB) ID: 1P9E) as shown in SEQ ID NO: 1, wherein the recombinant organophosphate-hydrolyzing enzyme has hydrolytic activity towards organophosphate insecticides.
(b) A recombinant carbamate-hydrolyzing enzyme having at least 80 percent sequence identity to the amino acid sequence of L-aminopeptidase-D-Ala-esterase/amidase (arylamidase) (MAH) (PDB ID: 1B65) as shown in SEQ ID NO: 2, wherein the recombinant carbamate-hydrolyzing enzyme has hydrolytic activity towards carbamate insecticides.
(c) A recombinant pyrethroid-hydrolyzing enzyme having at least 80 percent sequence identity to the amino acid sequence of pyrethroid-hydrolyzing carboxylesterase (EstA) (PDB ID: A4CCY) as shown in SEQ ID NO: 3, wherein the recombinant pyrethroid-hydrolyzing enzyme has hydrolytic activity towards pyrethroid insecticides.

The present embodiment further provides three recombinant bacterial strains using Escherichia coli strain BL21 (DE3) as a host cell that was transformed with each of the recombinant plasmid DNA vectors described above.

The present embodiment also discloses the cultivation conditions for the recombinant bacterial strains described above, including the medium composition, the induction condition, and methods for enzyme harvesting and enzyme activity assays.

Moreover, the present embodiment provides a formulation, comprising the three recombinant enzymes described above as its functional components, for hydrolyzing organophosphate, carbamate, and pyrethroid insecticides.

### BRIEF DESCRIPTION OF THE FIGURES

FIG. 1 illustrates a map of the 6,646-basepair expression vector pEL-OP, which is composed of a modified pET22b (+) with LamB signal sequence and a gene encoding MPH, to produce a recombinant enzyme methyl parathion hydrolase in a bacterial host cell.

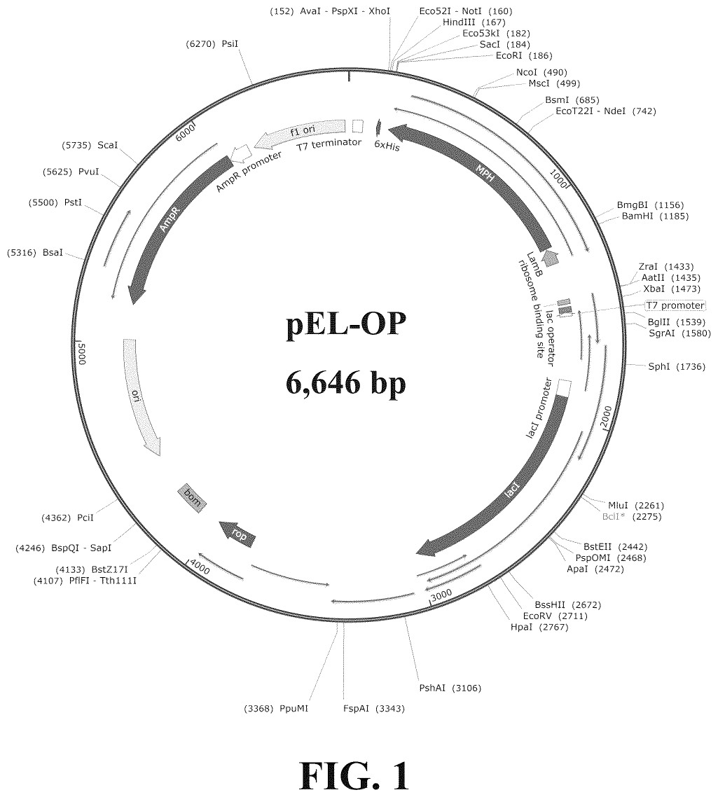

FIG. 2 illustrates a map of the 7,144-basepair expression vector pEL-CM, which is composed of a modified pET22b (+) with LamB signal sequence and a gene encoding MAH, to produce a recombinant enzyme L-aminopeptidase-D-Ala-esterase/amidase (arylamidase) in a bacterial host cell.

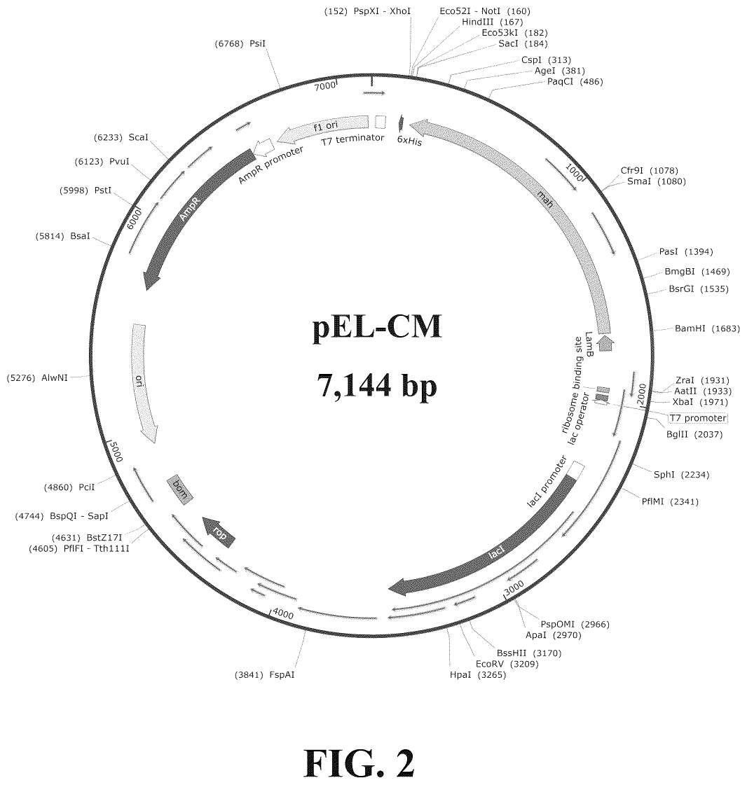

FIG. 3 illustrates a map of the 6,418-basepair expression vector pEL-PY, which is composed of a modified pET22b (+) with LamB signal sequence and a gene encoding EstA, to produce a recombinant enzyme pyrethroid-hydrolyzing carboxylesterase in a bacterial host cell.

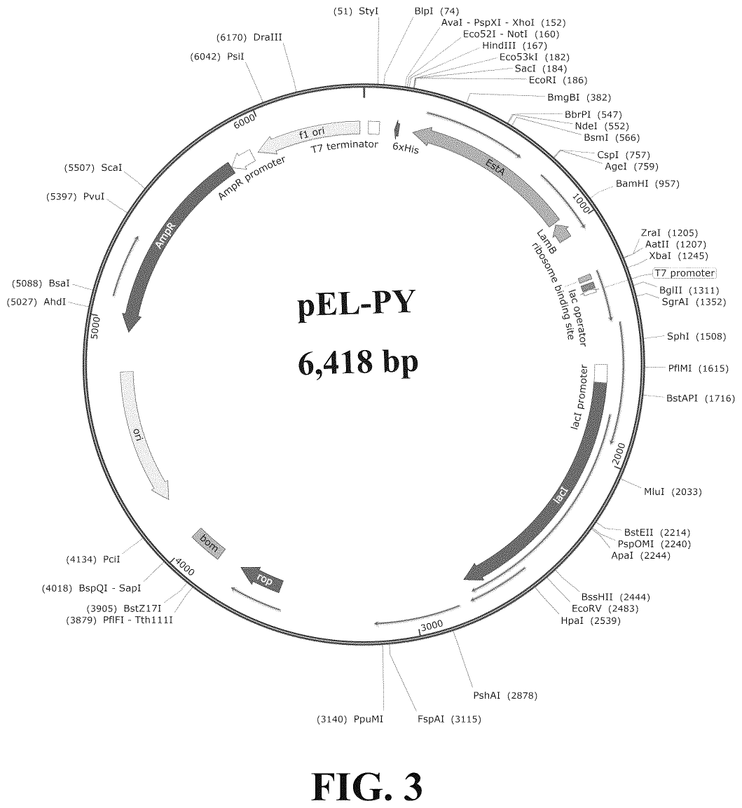

FIG. 4 depicts the protein expression of crude enzymes, extracted from the recombinant host cells during fermentation in a 5-liter fermenter from 0 to 16 hours, as shown in an assay using sodium dodecyl sulfate polyacrylamide gel electrophoresis (SDS-PAGE). The molecular weight of each recombinant enzyme is indicated, namely 34.8 kDa of methyl parathion hydrolase or MPH, 56.5 kDa of L-aminopeptidase-D-Ala-esterase/amidase or arylamidase, and 30 kDa of pyrethroid-hydrolyzing carboxylesterase.

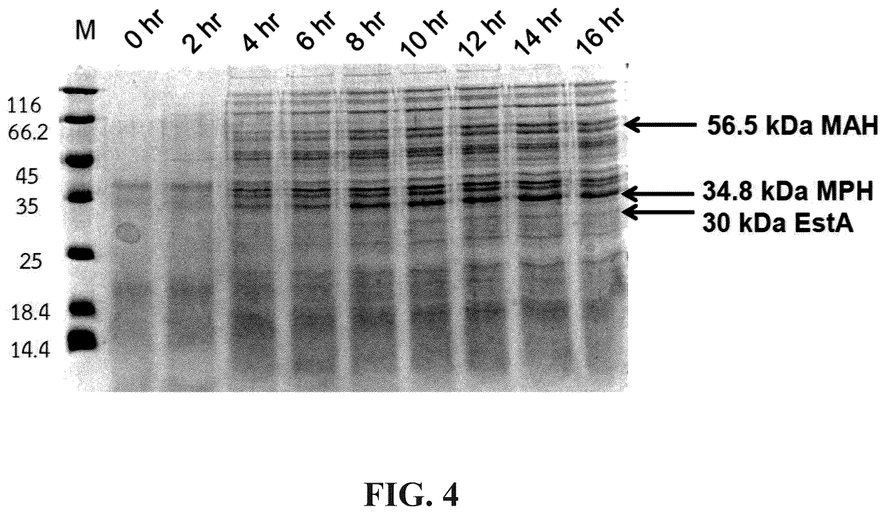

### DESCRIPTION OF THE INVENTION

As used herein, the singular forms “a”, “and”, and “the” comprise plural nouns, unless the context clearly indicates otherwise. Consequently, references to “a cell” comprise a plurality of cells, and references to “the protein” include one or more proteins and their equivalents. Unless otherwise specified, all technical and scientific words used in this document have the same meaning as commonly known by one of ordinary ability and skill in the art to which this disclosure belongs.

The present embodiment relates to the recombinant expression of the three enzymes which consist of the amino acid sequences, SEQ ID NO: 1, SEQ ID NO: 2, and SEQ ID NO: 3, in an Escherichia coli BL21 (DE3) host cell. The recombinant expression described above results in a cell with the ability to produce and secrete three recombinant enzymes, which ultimately leads to the production of a formulation containing these enzymes. Each of the three recombinant enzymes is capable of hydrolyzing a specific class of insecticide, namely organophosphate, carbamate, and pyrethroid. Thus, the aforementioned recombinant enzymes and formulation are useful for biodegrading and detoxifying organophosphate, carbamate, and pyrethroid insecticide residues present on the surface of fruits and vegetables, as well as in insecticide-contaminated environments.

The term “methyl parathion hydrolase” refers to an enzyme with the systematic name of aryl-triphosphate dialkyl-phosphohydrolase and the enzyme commission number, E. C. 3.1.8.1. This enzyme catalyzes the hydrolysis of organophosphorus compounds, including esters of phosphonic and phosphinic acids. The “recombinant methyl parathion hydrolase” of the present embodiment is a methyl parathion hydrolase enzyme, which is naturally expressed in bacteria other than the host cell of the invention, i.e. a heterologous methyl parathion hydrolase, the amino acid of which has not been modified or has been modified preferably by means of one or more deletions, insertions or substitutions. In a preferred embodiment, the recombinant methyl] parathion hydrolase is a naturally occurring methyl parathion hydrolase, derived from bacteria other than the host cell of the embodiment, preferably from Ochrobactrum sp.

The recombinant methyl parathion hydrolase, referred to in the present disclosure, comprises an amino acid sequence that is at least 80 percent identical to SEQ ID NO: 1. The said amino acid sequence of the recombinant methyl parathion hydrolase may be obtained from the amino acid sequence of methyl parathion hydrolase of a Gram-negative bacterium, such as Ochrobactrum sp., Stutzerimonas stutzeri, Pseudomonas putida, Pseudomonas sp., Caballeronia zhejiangensis, Stenotrophomonas sp., Achromobacter sp., Stenotrophomonas maltophilia, Burkholderia sp., and Cupriavidus sp.

The term “L-aminopeptidase-D-Ala-esterase/amidase or arylamidase” refers to an enzyme having the systematic name of D-stereospecific aminopeptidase, with the enzyme commission number, E.C. 3.4.11.19. This enzyme catalyzes the hydrolytic cleavage of an amide bond. Examples of the substrates with an amide group are D-amino acid amides, methy] esters, and amide pesticides, such as carbamate insecticides and herbicides, organophosphate insecticides with an amide group, and acetanilide herbicides. The “recombinant L-aminopeptidase-D-Ala-esterase/amidase or arylamidase” of the present embodiment is an L-aminopeptidase-D-Ala-esterase/amidase or arylamidase enzyme, which is naturally expressed in bacteria other than the host cell of the embodiment, i.e. a heterologous L-aminopeptidase-D-Ala-esterase/amidase or arylamidase, the amino acid of which has not been modified or has been modified preferably by means of one or more deletions, insertions or substitutions. In a preferred embodiment, the recombinant L-aminopeptidase-D-Ala-esterase/amidase or arylamidase is a naturally occurring enzyme derived from bacteria other than the host cell of the present embodiment, preferably from Ochrobactrum sp.

The recombinant L-aminopeptidase-D-Ala-esterase/amidase or arylamidase, referred to in the present disclosure, comprises an amino acid sequence that is at least 80 percent identical to SEQ ID NO: 2. The said amino acid sequence of the recombinant L-aminopeptidase-D-Ala-esterase/amidase or arylamidase may be obtained from the amino acid sequence of L-aminopeptidase-D-Ala-esterase/amidase or arylamidase of a Gram-negative bacterium, such as Ochrobactrum sp., Gemmobacter nanjingensis, and Starkeya sp.

The term “pyrethroid-hydrolyzing carboxylesterase” refers to an enzyme with the systematic name of pyrethroid-ester hydrolase and the enzyme commission number, E.C. 3.1.1.88. This enzyme catalyzes the hydrolysis of pyrethroid pesticides. The “recombinant pyrethroid-hydrolyzing carboxylesterase” of the present embodiment is a pyrethroid-hydrolyzing carboxylesterase enzyme, which is naturally expressed in bacteria other than the host cell of the embodiment, i.e. a heterologous pyrethroid-hydrolyzing carboxylesterase, the amino acid of which has not been modified or has been modified preferably by means of one or more deletions, insertions or substitutions. In a preferred embodiment, the recombinant pyrethroid-hydrolyzing carboxylesterase is a naturally occurring pyrethroid-hydrolyzing carboxylesterase derived from bacteria other than the host cell of the present embodiment, preferably from Bacillus sp.

The recombinant pyrethroid-hydrolyzing carboxylesterase, referred to in the present disclosure, comprises an amino acid sequence that is at least 80 percent identical to SEQ ID NO: 3. The said amino acid sequence of the recombinant pyrethroid-hydrolyzing carboxylesterase may be obtained from the amino acid sequence of pyrethroid-hydrolyzing carboxylesterase of a Gram-positive bacterium, such as Bacillus sp., Bacillus cereus, Bacillus thuringiensis, Bacillus wiedmannii, and Bacillus tropicus.

The term “signal sequence” refers to an N-terminal, short amino acid sequence that mediates the transfer of any attached or fused polypeptide or protein or recombinant protein toward the secretory pathway of the cell or the host cell. The signal sequence of the present embodiment is a 75-base nucleotide sequence encoding LamB (maltoporin or phage lambda receptor) amino acid sequence that is identical to a Gram-negative bacterial maltoporin and may be obtained from Escherichia coli, Salmonella enterica, Klebsiella pneumoniae, or Shigella sonnei.

The terms “nucleic acid construct” and “gene construct” are used interchangeably herein and refer to a nucleic acid molecule that is isolated from a naturally occurring gene, synthesized according to a natural gene sequence, or modified to contain segments of nucleic acids in a particular manner. “Nucleic acid construct” and “gene construct” are synonymous with the term, “expression cassette”, when the nucleic acid construct contains the control sequences necessary for the expression of a coding sequence of the recombinant enzyme.

The terms “expression vector” and “expression construct” are used interchangeably herein. An expression vector is a plasmid DNA vector designed for gene expression by accommodating mechanisms for protein synthesis to produce the protein encoded by the gene, and thus is used to introduce a specific gene into the host cell. The expression vector, described in the present disclosure, was constructed using the expression vector pET22b (+) with a modified signal sequence, in which a nucleotide sequence encoding PelB was deleted and replaced with a nucleotide sequence encoding LamB to engender a more efficient secretory recombinant protein. The expression construct is operably linked to additional nucleotides, which provide for its expression, and introduced into the host cell so that the expression vector is maintained as a chromosomal integrant or self-replicating extra-chromosomal vector.

The expression vector is used to generate the recombinant methyl parathion hydrolase (MPH) by means of any method currently accepted in the field. The methods described in this disclosure include the codon optimization, gene synthesis, cloning of the gene encoding methyl parathion hydrolase (MPH) according to SEQ ID NO: 1 into the modified expression vector creating pEL-OP, and transformation into bacterial host cells. The transformation of the expression vector into the host cell, Escherichia coli DH5-alpha, is used to multiply the number of expression vectors and evaluate the expression construct's accuracy. Subsequently, the transformation of the expression vector into the host cell, Escherichia coli BL21 (DE3), followed by the cultivation of the said host cell under conditions, described in the present disclosure, result in the production of the secreted methyl parathion hydrolase (MPH).

The expression vector is used to generate the recombinant L-aminopeptidase-D-Ala-esterase/amidase or arylamidase (MAH) by means of any method currently accepted in the field. The methods described in this disclosure include the codon optimization, gene synthesis, cloning of the gene encoding L-aminopeptidase-D-Ala-esterase/amidase or arylamidase (MAH) according to SEQ ID NO: 2 into the modified expression vector creating pEL-CM, and transformation into bacterial host cells. The transformation of the expression vector into the host cell, Escherichia coli DH5-alpha, is used to multiply the number of expression vectors and evaluate the expression construct's accuracy. Subsequently, the transformation of the expression vector into the host cell Escherichia coli BL21 (DE3), followed by the cultivation of the said host cell under the conditions described in the present disclosure, result in the production of the secreted L-aminopeptidase-D-Ala-esterase/amidase or arylamidase (MAH).

The expression vector is used to generate the recombinant pyrethroid-hydrolyzing carboxylesterase (EstA) by means of any method currently accepted in the field. The methods described in this disclosure include the codon optimization, gene synthesis, cloning of the gene encoding pyrethroid-hydrolyzing carboxylesterase (EstA) according to SEQ ID NO: 3 into the modified expression vector creating pEL-PY, and transformation into bacterial host cells. The transformation of the expression vector into the host cell, Escherichia coli DH5-alpha, is used to multiply the number of expression vectors and evaluate the expression construct's accuracy. Subsequently, the transformation of the expression vector into the host cell Escherichia coli BL21 (DE3), followed by the cultivation of the said host cell under the conditions, described in the present disclosure, result in the production of the secreted pyrethroid-hydrolyzing carboxylesterase (EstA).

The host cell, described in the present disclosure, expresses a functional recombinant enzyme that comprises methyl parathion hydrolase (MPH), L-aminopeptidase-D-Ala-esterase/amidase or arylamidase (MAH), and pyrethroid-hydrolyzing carboxylesterase (EstA). Such a host cell is capable of secreting the recombinant enzyme into the extracellular medium. The term “functional” means that the expressed enzyme retains its capacity to hydrolyze its insecticide substrates, i.e. organophosphate, carbamate, and pyrethroid, respectively. Each enzyme activity can be measured by means of any suitable method known in the art to assess the hydrolytic activity towards each substrate group, preferably using the methods described below for the present embodiment.

The first aspect of the embodiment refers to the three expression vectors, namely pEL-OP, pEL-CM, and pEL-PY, preferably constituting a set. Moreover, the three recombinant enzymes are expressed by the host cell of the invention. Preferably, the three aforementioned recombinant enzymes consist of the enzyme with the amino acid sequences of SEQ ID NO: 1, SEQ ID NO: 2, and SEQ ID NO: 3 as shown in Table 1.

| SED | Recombinant | |
|---|---|---|
| ID NO. | enzyme | Sequence |
| SEQ ID | Methyl parathion | MPLKNRLLARLSCVAAVVAATAAVAPLTLVSTAHAAAPQVRTSAPGYYRMLLGDFEITALSDG |
| NO: 1 | hydrolase | TVALPVDKRLNQPAPKTQSALAKYFQKAPLETSVTGYLVNTGSKLVLVDTGAAGLFGPTLGRLA |
| | | ANLKAAGYQPEQVDEIYITHMHPDHVGGLMVGEQLAFPNAVVRADQKEADFWLSQTNLDKAP |
| | | DDSKGFFKGAMASLNPYVKAGKFKPFSGNTDLVPGIKALASHGHTAGHTTYVVESQGQKLALL |
| | | GDLILVAAVQFDDPSVTNQLDSDSKSAAVERKKAFADAAKGGYLIAAAHLSFPGIGHIRAEGKG |
| | | YRFVPVNYSVVNPK |
| SEQ ID | L-aminopeptidase- | MERSDLDYASATEIARLVRTRQISAADVTEHAISRIEARNGSLNAFVYTDFEQARSRAKDLDTRIS |
| NO: 2 | D-Ala esterase/ | AGEDVGPLAGVPTAIKDLFNFYPGWPSTLGGIRCLRDFKLDVKSRYATKMEEAGAVVLGITNSP |
| | amidase | VLGFRGTTDNDLYGPTRNPFDLSRNSGGSSGGTSAAVADGLLPIGDGTDGGGSIRIPAAWCHVFG |
| | or Arylamidase | FQASPGRIPLAIRPNAFGAAAPFIYEGPITRTVEDAALAMSVLAGSDPADPFSLNDRLDWLGAVD |
| | | QPITSLRIGFTPDFGGFPVEPAVAATIAHAVRAFEQAGAKIVPLKLDFGYTHDELSQLWCRMISQG |
| | | TIAVVDSFAENGLHLEPDFPAPVMEWAQKAKNATPLDLHRDQVMRTKVYDVLNAAFSQVDLIA |
| | | GPTTTCLPTPNGERGMTVGPSEIAGTPINRLIGFCPTFLTNFTGNPAASLPAGLADGLPVGLMLIG |
| | | PRRDDLTVLSASAAFERVQPWADSYRIPAARPLGSQ |
| SEQ ID | pyrethroid- | MMKLASPKPFTFEGGDRAVLLLHGFTGNSADVRMLGRFLEKKGYTCHAPIYKGHGVPPEELVH |
| NO: 3 | hydrolyzing | TGPEDWWQDVTEAYQLLKDKGFEKIAVVGLSLGGVFSLKLGYTVPVLGVVPMCAPMYIKSEET |
| | carboxylesterase | MYQGILAYAREYKKREQKSPEQIEQEMLEFQKTPMNTLKALQQLIADVRNNVDMIYAPTFVVQ |
| | | ARHDEMINTDSANIIYNGVESTLKDIKWYEDSTHVITLDKQRDELHEDVYNFLEQLDW |

The cultivation medium and conditions of the host cell for producing the recombinant enzyme complies with methods well known in the art. For example, the host cell is cultivated by shake flask cultivation and small-scale or large-scale fermentation (including continuous, batch, or fed-batch fermentations) in the laboratory or an industrial bioreactor, performed in a suitable medium and under conditions that allow the recombinant enzyme to be expressed. The cultivation takes place in a suitable nutrient medium comprising carbon sources, nitrogen sources, inorganic salts, and an antibiotic. The seed culture cultivation conditions described in this disclosure involves growing the host cell producing each recombinant enzyme with 1 to 5 percent (volume by volume) inoculum in a 100-microgram per milliliter ampicillin-containing Luria Bertani (LB) medium for 16 hours at 35 to 37 degrees Celsius and with 200 rpm shaking speed. The seed culture of each recombinant host strain is grown separately or together in the volume ratio of 1:1:1.

The second aspect of the embodiment relates to the cultivation and induction of the recombinant host cell for producing the enzyme. The cultivation for the enzyme production stage, described in this disclosure, entails the growth of the recombinant host cell in SPY medium for generating each recombinant enzyme under the indicated conditions. The SPY medium, which is prepared at pH 7 to 8, contains a composition with one or more constituents, comprising 1 to 10 grams per liter of soy peptone, soybean powder, or soy protein; 1 to 10 grams per liter of yeast extract; and/or 5 to 10 grams per liter of sodium chloride. Included in the said composition are one or more coenzymes, comprising a 1 to 10 millimolar trace element of magnesium chloride, cobalt chloride, di-cobalt chloride, copper chloride, manganese chloride, and/or ferric chloride. At least one constituent for stimulating enzyme expression is also incorporated into the composition, wherein the constituent comprises 1 to 10 millimolar of galactose or 5 to 20 millimolar of lactose. Adding 100 micrograms per milliliter of ampicillin to the composition is optional but preferred. At this stage, the seed culture can be inoculated in one of two ways: either with 5 percent (volume by volume) of each of the three recombinant host strains grown separately, or with 15 percent (volume by volume) of the three recombinant host strains grown together in the volume ratio of 1:1:1 under the conditions described above. The recombinant cells are grown for 3 to 4 hours at 35 to 37 degrees Celsius and at 200 rpm shaking speed, until the cell optical density, as measured with a spectrophotometer at a wavelength of 600 nanometers, reaches 0.6 to 0.8.

The second aspect further includes the induction conditions for enzyme expression and production. The induction conditions of the recombinant host cell for producing the enzyme, described in the present disclosure, involve adding an enzyme expression-inducing composition with one or more constituents, namely 1 to 10 millimolar of galactose or 5 to 20 millimolar of lactose, alongside the enzyme secretion-enhancing solution, comprising 5 to 20 percent (weight by volume) of sucrose, 1 to 2 percent (weight by volume) of glycine, and 1 to 2 percent (weight by volume) of Tween-20 in the growing recombinant cells mentioned above. The recombinant cells are further grown for at least 16 to 20 hours at 30 to 37 degrees Celsius and at 200 rpm shaking speed. The enzyme activities are analyzed at intervals using the methods described in the examples below. Determining protein expression by methods well known in the field, e.g. sodium dodecyl sulfate polyacrylamide gel electrophoresis (SDS-PAGE), is optional. At the indicated time, the recombinant cells are removed by centrifugation or 0.22-micron membrane filtration at 4 to 10 degrees Celsius. The recovery of the crude enzyme product is accomplished using membrane filtration.

The third aspect of the embodiment involves the formulation of the three recombinant enzymes, used as functional components, in liquid form for creating the final product. Said formulation is prepared in a 10-millimolar glycine buffer at pH 7.5-8.5 and consists of 30 to 50 percent by volume of methyl parathion hydrolase, 5 to 15 percent by volume of L-aminopeptidase-D-Ala-esterase/amidase (Arylamidase), and 5 to 15 percent by volume of pyrethroid-hydrolyzing carboxylesterase, with 30 to 40 percent by volume of glycerol as a stabilizer.

### EXAMPLES

### Example 1. The Enzymatic Activity Assay of Methyl Parathion Hydrolase by Means of Hydrolysis of Organophosphate Substrate

The methyl parathion hydrolase (E.C. 3.1.8.1), having the amino acid sequence shown in SEQ ID NO: 1, is a hydrolytic enzyme catalyzing the hydrolysis of organophosphate compounds, including esters of phosphonic and phosphinic acids. Using a specific substrate for methyl parathion hydrolase, this enzyme's activity is measured using the same procedures as glucoamylase activity (amyloglucosidase activity), in accordance with the guidelines in the Combined Compendium of Food Additive Specification and Food Chemical Codex.

Methyl parathion hydrolase activity is determined using methyl paraoxon (O,O-dimethyl O-(4-nitrophenyl) phosphate; CAS number: 950-35-6) as a substrate that is comparable to an organophosphate compound. For this assay, the enzymatic reaction mixture (750 microliter at final volume), containing 50 to 100 millimolar Tris-HCl buffer (pH 8.0), 0.1 millimolar cobalt chloride, 0.8 millimolar methyl paraoxon, and a proper amount of the respective enzyme (with a protein concentration of approximately 0.8+0.2 milligrams per milliliter), are incubated for 5 minutes at 37±2 degrees Celsius. The amount of para-nitrophenol released as a hydrolytic product (a yellow-colored product) is measured using a spectrophotometer at the absorption wavelength of 410 nanometers. The enzyme activity is calculated using either a para-nitrophenol standard curve or the equation shown below, with the molar extinction coefficient (ε) of 11,933 M−1 cm−1. One unit of methyl paraoxon hydrolyzing activity is defined as the amount of enzyme needed to release one micromole of para-nitrophenol per minute. Protein concentration of the enzyme was determined by either the Lowry protein assay method (Thermo Scientific™ Pierce 23240) or the BCA method (AppliChem, A7787 0500), and calculated with the protein standard curve using Bovine serum albumin as a protein standard. The specific activity of the enzyme is defined as a unit of enzyme per milligram of protein. The production of the enzyme in the shake-flask condition yields 10 to 15 units per milliliter of the recombinant methyl parathion hydrolase, whereas a 5-liter to 300-liter fermenter production yields 45 to 50 units per milliliter.

$$Methyl parathion hydrolase activity ( unit per mL ) = \frac{\begin{matrix}( absorption value at 410 nm ) xX \\ ( reaction volume , mL ) x ( dilution factor )\end{matrix}}{\begin{matrix}( molar extinction coefficient ) x \\ ( enzyme volume , mL ) X ( incubation time , min )\end{matrix}}$$

$$Specific enzyme activity ( unit per mg of protein ) = \frac{Unit per mL}{Mg of protein per ML}$$

### Example 2. The Enzymatic Activity Assay of L-Aminopeptidase-D-Ala-Esterase/Amidase or Arylamidase by Means of Hydrolysis of Carbamate Substrate

The L-aminopeptidase-D-Ala-esterase/amidase or arylamidase (E.C. 3.4.11.19), having the amino acid sequence shown in SEQ ID NO: 2, is a hydrolytic enzyme catalyzing the hydrolytic cleavage of an amide bond of a substrate with an amide group, including D-amino acid amides, methyl esters, and amide pesticides, such as carbamate insecticides and herbicides, organophosphate insecticides with an amide group, and acetanilide herbicides. The enzyme activity assay is conducted following the reference method described in Ozturk et al. (2016) Environmental Microbiology 18 (12), 4878-4887 (doi.org/10.1111/1462-2920.13409) and Zhang et al. (2019) Ecotoxicology and Environmental Safety 15 (167), 122-129 (doi: 10.1016/j.ecoenv.2018.09.127.)

For L-aminopeptidase-D-Ala-esterase/amidase or arylamidase activity assay, the enzymatic reaction mixture (1 milliliter at final volume), containing 10 to 50 millimolar glycine buffer (pH 8.0), 0.8 to 1.0 millimolar carbaryl as a representative of a carbamate insecticide, anda proper amount of the respective enzyme (with a protein concentration of approximately 0.8±0.2 milligrams per milliliter), are incubated for 15 minutes at 37±2 degrees Celsius. The amount of carbaryl hydrolyzed or the amount of 1-napthol released as a hydrolytic product is measured using a reverse phase high performance liquid chromatography (HPLC), equipped with a photodiode array detector having a C18 column (4.6-millimeter internal diameter, and 250-millimeter length) and an acetonitrile-water mobile phase (70:30, volume by volume) with a flow rate of 1 milliliter per minute. The monitored wavelengths are 212 and 280 nanometers, at which the retention time of carbaryl and 1-napthol are 10.30±0.09 and 11.66±0.06 minutes, respectively. The decrease in substrate concentration (carbaryl; Merck 32055; CAS number 63-25-2) or the formation of a product (1-napthol; Merck N100; CAS number 90-15-3) is determined from the HPLC peak height ratio relative to its standard calibration curve. Protein concentration of the enzyme was determined by either the Lowry protein assay method (Thermo Scientific™ Pierce 23240) or the BCA method (AppliChem, A7787 0500), and calculated with the protein standard curve using Bovine serum albumin as a protein standard. The specific activity of the enzyme is defined as a unit of enzyme per milligram of protein. The production of the enzyme in the shake-flask condition yields 3 to 5 units per milliliter of the recombinant arylamidase (MAH), whereas a 5-liter to 300-liter fermenter production yields 10 to 15 units per milliliter.

### Example 3. The Enzymatic Activity Assay of Pyrethroid-Hydrolyzing Carboxylesterase by Means of Hydrolysis of Pyrethroid Substrate

The pyrethroid-hydrolyzing carboxylesterase (E.C. 3.1.1.88), having the amino acid sequence shown in SEQ ID NO: 3, is an enzyme capable of catalyzing the hydrolysis of pyrethroid pesticides. The enzyme activity is determined using a colorimetric assay, conducted following the reference method described in Pengpumkiat et al. (2020) Sensors 2020, 20, 4107 (doi: 10.3390/s20154107).

The pyrethroid-hydrolyzing carboxylesterase activity is determined using a substrate of cypermethrin (Merck 239900; CAS number 52315-07-8) as a representative of a pyrethroid insecticide. The enzymatic reaction mixture (1 milliliter at final volume), containing 10 to 50 millimolar glycine buffer (pH 8.6), 0.8 to 1.0 millimolar of cypermethrin, and a proper amount of the respective enzyme (with a protein concentration of approximately 0.8±0.2 milligrams per milliliter), are incubated for 5 minutes at 37±2 degrees Celsius. Then, ammonium acetate (10 percent, volume by volume) and ninhydrin solution in ethanol (4 percent, volume by volume) are added to the mixture to react with the free cyanide, released from the hydrolysis, forming a purple-colored product. The quantitative analysis of the purple-colored product is conducted using a spectrophotometer at the absorption wavelength of 570 nanometers. The enzyme activity is calculated from the amount of pyrethroid hydrolytic product using the cyanide standard calibration curve. Protein concentration of the enzyme was determined by either the Lowry protein assay method (Thermo Scientific™ Pierce 23240) or the BCA method (AppliChem, A7787 0500), and calculated with the protein standard curve using Bovine serum albumin as a protein standard. The specific activity of the enzyme is defined as a unit of enzyme per milligram of protein. The production of the enzyme in the shake-flask condition yields 6 to 10 units per milliliter of the recombinant pyrethroid-hydrolyzing carboxylesterase, whereas a 5-liter to 300-liter fermenter production yields 15 to 20 units per milliliter.

The invention claimed is:

1. A set of recombinant plasmid vectors for producing recombinant enzymes capable of hydrolyzing organophosphate, carbamate, and pyrethroid insecticides, the set of recombinant plasmid vectors comprising:
a first recombinant vector, having a gene for encoding a first recombinant enzyme, having an amino acid sequence of at least 80% identity to SEQ ID NO: 1;
a second recombinant vector, having a gene for encoding a second recombinant enzyme, having an amino acid sequence of at least 80% identity to SEQ ID NO: 2; and
a third recombinant vector, having a gene for encoding a third recombinant enzyme, having an amino acid sequence of at least 80% identity to SEQ ID NO: 3; and
wherein each of the first recombinant plasmid vector, second recombinant plasmid vector, and third recombinant plasmid vector is separately transformed into individual bacterial host cells; and wherein the individual bacterial host cells are modified to express the recombinant enzymes.

2. The set of recombinant plasmid vectors according to claim 1, wherein the first recombinant enzyme having the amino acid sequence of at least 80% identity to SEQ ID NO: 1 is a recombinant methyl parathion hydrolase.

3. The set of recombinant plasmid vectors according to claim 1, wherein the second recombinant enzyme having the amino acid sequence of at least 80% identity to SEQ ID NO: 2 is a recombinant L-aminopeptidase-D-Ala-esterase/amidase or arylamidase.

4. The set of recombinant plasmid vectors according to claim 1, wherein the third recombinant enzyme having the amino acid sequence of at least 80% identity to SEQ ID NO: 3 is a recombinant pyrethroid-hydrolyzing carboxylesterase.

5. The set of recombinant plasmid vectors according to claim 1, wherein each of the individual bacterial host cells is Escherichia coli strain BL21 (DE3).

6. A method for producing the recombinant enzymes capable of hydrolyzing organophosphate, carbamate, and pyrethroid insecticides, wherein the method comprises: culturing the individual bacterial host cells according to claim 5 in a medium and collecting the recombinant enzymes from the medium.

7. A formulation for creating a final product, the formulation comprising the recombinant enzymes capable of hydrolyzing organophosphate, carbamate, and pyrethroid insecticides, wherein the recombinant enzymes are used as functional components of said formulation and produced by the individual bacterial host cells according to claim 5.
